(19) United States
(12) Patent Application Publication
(10) Pub. No.: US 2022/0080346 A1
(43) Pub. Date: Mar. 17, 2022

(54) SYSTEM AND METHOD FOR CLEANING A VEHICLE PASSENGER COMPARTMENT

(71) Applicant: RUBIX S&I, Toulouse (FR)

(72) Inventor: Jean-Christophe Mifsud, Goudourville (FR)

(73) Assignee: RUBIX S&I, Toulouse (FR)

(21) Appl. No.: 17/423,091

(22) PCT Filed: Feb. 5, 2020
(86) PCT No.: PCT/EP2020/052822

§ 371 Date: Jul. 14, 2021

(30) Foreign Application Priority Data
Feb. 7, 2019 (FR) 1901215

(51) Int. Cl. B01D 53/00; B01D 53/30; B60H 1/00; B60H 1/22

(52) U.S. Cl. CPC B01D 53/005; B01D 53/30; B60H 1/2226; B60H 1/008; B60H 1/00878; B60H 1/00742

(57) ABSTRACT

Vehicle comprising a passenger compartment and a system for cleaning the passenger compartment comprising at least one measuring member for measuring the level of dirtiness of said passenger compartment, at least one heating member configured to heat the ambient air of the passenger compartment to a temperature higher than 40° C. in order to desorb the impurities in the passenger compartment, at least one ventilation member configured to remove the ambient air and the desorbed impurities to outside the passenger compartment and at least one control member configured to activate the heating member if the measured level of dirtiness is above a predetermined maximum level of dirtiness.

### TECHNICAL FIELD

The present invention relates to the field of cleaning a vehicle passenger compartment and is more particularly directed to a system and a method for cleaning ambient air and elements of the passenger compartment of said vehicle.

### BACKGROUND

In a known manner, an automobile vehicle allows its users to move from one location to another. A vehicle comprises a passenger compartment which is defined as the part of the vehicle reserved for users. This passenger compartment comprises various passenger compartment elements to provide comfort and safety to the users (seat, sun visor, etc.). During a journey, the passenger compartment is dirtied by various impurities such as odors of sweat, cigarettes or food of its users, the odors of pets or volatile organic compounds, known to those skilled in the art under the abbreviation “VOC”, such as polluting particles, dust or pollen coming from outside. All of these impurities permeate both ambient air in the passenger compartment and elements of the passenger compartment and adversely affect the comfort of the vehicle users. This is particularly detrimental for automobile vehicles that are subject to a high occupancy rate, namely collective vehicles (buses, etc.) and shared vehicles (autonomous cars, rental cars, etc.).

Currently, to clean the passenger compartment of a vehicle with a high occupancy rate, the vehicle is periodically moved to a cleaning station where impurities in the passenger compartment are discharged by means of an outer cleaning device, such as a vacuum cleaner. This method has the drawback of being expensive, since it requires, on the one hand, moving the vehicle to a dedicated cleaning station and, on the other hand, manpower to perform cleaning. Moreover, such a cleaning is complex to plan because the passenger compartment is not dirtied in the same way depending on the frequency of use and the occupancy rate of the vehicle. While early cleaning is a waste of time and money, late cleaning can cause discontent among vehicle users.

A method for cleaning the passenger compartment of a vehicle subject to a high occupancy rate is known in the prior art, from patent application EP3278817A1, which teaches to inject hot air combined with a disinfectant into the closed passenger compartment of the vehicle by means of a duct coming from an external system. This method has the advantage of not requiring manpower to perform cleaning but does not solve the other drawbacks mentioned in the previous paragraph (required movement of the vehicle and complex planning of cleaning). Further, this method has the drawback of only discharging impurities present in the air of the passenger compartment.

Alternatively, a method for cleaning the passenger compartment of an autonomous vehicle is known from patent application US2018/0126960A1, in which said autonomous vehicle is moved to a cleaning station when sensors for the level of insalubrity present in the passenger compartment indicate this. This method has the advantage of being able to accurately plan cleaning of the vehicle, but does not enable other drawbacks to be solved (required movement of the vehicle, use of manpower to perform cleaning, etc.).

Therefore, there is a need for a system and method for cleaning the passenger compartment of a vehicle subject to a high occupancy rate, such as an autonomous vehicle, that is inexpensive, that can be performed when the state of cleanliness of said vehicle is unsatisfactory, and that effectively treats impurities.

Although the invention was originally conceived for an automobile vehicle, it is more generally applicable to any vehicle comprising a passenger compartment, in particular, a train, an airplane, a boat, etc.

### SUMMARY

To this end, the invention relates to a vehicle comprising a passenger compartment and a system for cleaning the passenger compartment comprising at least one member for measuring the level of insalubrity N of said passenger compartment, at least one heating member configured to heat ambient air of the passenger compartment to a temperature above 40° C. in order to desorb impurities from the passenger compartment, at least one ventilation member configured to discharge ambient air and desorbed impurities outside the passenger compartment and at least one control member configured to activate the heating member if the measured level of insalubrity N is greater than a predetermined maximum level of insalubrity NET.

The invention is remarkable in that the cleaning system is internal to the vehicle, which avoids moving the vehicle to a cleaning station, and allows the passenger compartment to be purified autonomously without manpower. This cleaning system is moreover effective, because it is included in the passenger compartment, it is as close as possible to the impurities to be treated. This position also advantageously facilitates installation and maintenance of the cleaning system. This is particularly advantageous in the case of shared vehicles.

According to one aspect of the invention, the heating member is configured to heat ambient air of the passenger compartment to a temperature above 45° C., preferably 50° C. Advantageously, most of the impurities present in the passenger compartment are desorbed at this temperature, that is they are extracted from elements of the passenger compartment and turn into a vapor phase, which facilitates their extraction by ventilation.

According to another aspect of the invention, the heating member is configured to heat ambient air of the passenger compartment to a temperature below 80° C., so as not to damage elements of said compartment.

Preferably, the vehicle comprises at least one passenger compartment element comprising at least one heating member configured to heat the passenger compartment element to a temperature above 40° C. to form a heating passenger compartment element to desorb impurities from the passenger compartment element. Advantageously, the cleaning system not only allows ambient air of the passenger compartment but also the passenger compartment elements to be cleaned, in order to remove impurities collected from the passenger compartment element and which are stored therewithin.

According to one aspect of the invention, the heating member is integrated into the passenger compartment element, so as to be able to effectively clean said passenger compartment element internally and not superficially. Furthermore, when the heating member is integrated, the external appearance of the element is not changed, which is advantageous in terms of comfort and aesthetics.

According to another aspect of the invention, the heating member is positioned at the surface of the passenger compartment element, so as to be able to effectively clean the superficial part of said passenger compartment element, which is particularly exposed to sources of impurities. These two installation configurations advantageously allow optimal cleaning of the passenger compartment depending on the type of passenger compartment element.

Preferably, the heating member is integrated into or positioned at the surface of a passenger compartment element comprising polyurethane foam or polyvinyl chloride or acrylonitrile butadiene styrene or polypropylene or polyethylene or polyester or an alloy of these thermoplastics, so as to improve heating and desorption. Preferably, the heating member is integrated into the volume of the passenger compartment element.

Preferentially, at least one heating passenger compartment element is a seat, in order to clean impurities primarily from the users' clothing, such as sweat.

Preferably, the vehicle comprises a plurality of heating passenger compartment elements for overall cleaning of the passenger compartment.

Preferentially, at least one heating passenger compartment element is the floor lining, in order to clean impurities primarily from the users' footwear, such as dirt.

Preferentially, at least one heating passenger compartment element is the roof lining, in order to clean impurities such as VOCs and odors, of cigarette smoke or food for example.

Preferentially, at least one heating passenger compartment element is the sidewall lining, in order to clean impurities primarily from users or heavy toxic substances.

Preferentially, at least one heating passenger compartment element is a sun visor, in order to clean impurities primarily from users, the vehicle, or external pollution.

Preferentially, at least one heating passenger compartment element is the instrument panel, in order to clean impurities mainly from users, the vehicle or external pollution.

Preferentially, at least one heating passenger compartment element is the lining of a door, in order to clean impurities primarily from users, the vehicle or external pollution.

Preferably, the heating member is in the form of a coil-shaped heating resistor. Preferably, the heating member is powered in a wired or wireless manner, for example, by induction. A heating member in such a form makes it possible to heat its vicinity homogeneously and effectively and thus to clean this vicinity overall and not partially. A wireless heating member allows greater freedom of positioning in a passenger compartment element, which is then removable from said passenger compartment.

According to one aspect of the invention, the measuring member is in the form of a gas sensor, such as a metal oxide sensor, and records the level of insalubrity N by the record of the presence of volatile toxic substances such as nitrogen dioxide N₁ and/or benzene-toluene-xylene N₂ and/or ozone N₃ and/or formaldehyde N₄ and/or any other volatile toxic substances, such as VOCs. This record of a plurality of data N₁-N₄ enables a reliable and objective measurement of the level of insalubrity N.

Preferentially, the predetermined maximum level of insalubrity N_{ET} is obtained from data banks on the maximum allowed presence of volatile toxic substances in the passenger compartment of the vehicle. Advantageously, the predetermined maximum level of insalubrity N_{ET} is thus a relevant comparison means because it is based on the type of impurities found in the vehicle.

Preferably, the power of the ventilation member is adapted to the level of insalubrity N and to the volume of the passenger compartment so as to rapidly discharge ambient air-suspended impurities and to minimize power consumption.

According to one aspect of the invention, the vehicle comprises at least one electric charging device such as an electric battery, so that the measuring member, control member, heating member and ventilation member are electrically powered by a system internal to the vehicle, said electric charging device operating regardless of whether the vehicle being in the running or stopped state.

Preferably, the cleaning system comprises at least one filtering member configured to treat desorbed impurities as they are discharged to outside of the passenger compartment. Advantageously, this cleaning system does not release potentially harmful impurities into the atmosphere.

Preferentially, the filtering member is in the form of a mechanical filter, in particular a porous filter which absorbs and retains impurities, such as an activated carbon filter, or in the form of a chemical filter which destroys molecules of impurities, such as a photocatalysis or ozonolysis filter, these two types of filter being effective for the types of impurities present in the passenger compartment.

The invention also relates to a method for cleaning the passenger compartment of the vehicle of the invention, comprising: a step of measuring the level of insalubrity N of the passenger compartment by means of the measuring member, a step of activating the heating step when the measured level of insalubrity N is greater than a predetermined maximum level of insalubrity N_{ET} by means of the control member, a step of heating the passenger compartment in order to heat ambient air of the passenger compartment and thus to desorb impurities present by means of the heating member and a step of ventilating the passenger compartment in order to discharge ambient air and impurities consumed outside the passenger compartment by means of the ventilation member.

Advantageously, this method makes it possible to clean the passenger compartment of a vehicle without moving it to a cleaning center, the entire cleaning system required to carry out the method being internal to the vehicle. Moreover, this method is autonomous so that it does not require any manpower. Furthermore, this method allows to detect a favorable moment to carry out cleaning, avoiding an unnecessary early cleaning and a late cleaning being a source of discontent for the occupants.

Preferably, the heating step of this method is implemented for a duration adapted to the level of insalubrity N and the volume of the passenger compartment so as to desorb impurities and minimize power consumption.

Preferentially, the duration of the heating step is adapted to desorb all impurities while minimizing power consumption.

Preferentially, during the activation step, the control member activates the heating step when there are no more users in the passenger compartment (NOP state), as a safety and comfort measure for the occupants (risk of discomfort, burning, bad odors, etc.).

Preferentially, the control member activates the heating step when the passenger compartment is closed (CLOSED state), that is the doors and windows are closed, in order to improve efficiency of the method.

Preferably, the ventilation step is implemented until the level of insalubrity N is below the predetermined maximum level of insalubrity N_{ET}. Advantageously, the ventilation step is optimized for time and energy cost to discharge impurities.

Preferably, the ventilation step is performed once the heating step is completed in order to avoid heat loss.

According to one aspect of the invention, the method for cleaning the passenger compartment of the vehicle comprises a step of filtering impurities consumed by means of the filtering member. Advantageously, this step makes it possible to avoid releasing potential harmful impurities into the atmosphere.

Preferentially, the filtering step is implemented during the ventilation step to save time.

### BRIEF DESCRIPTION OF THE DRAWINGS

The invention will be better understood upon reading the following description, given by way of example only, and referring to the appended drawings given as non-limiting examples, in which identical references are given to similar objects and in which:

FIG. 1 is a schematic side representation of an automobile vehicle according to the invention,

FIG. 2 is a schematic profile representation of an automobile vehicle according to the invention,

FIG. 3 is a functional schematic representation of the system for cleaning a vehicle passenger compartment according to the invention,

FIG. 4 is a schematic representation of a heating member of the cleaning system and

FIG. 5 is a schematic representation of the steps of the method for cleaning a vehicle passenger compartment according to the invention.

It should be noted that the figures set out the invention in detail for implementing the invention, said figures can of course be used to better define the invention if necessary.

### DETAILED DESCRIPTION

With reference to FIGS. 1-5, there are schematically represented a system and a method for cleaning a passenger compartment of an automobile vehicle V by internal heating according to one embodiment of the invention. As previously indicated, although the invention was originally created for an automobile vehicle, it is more generally applicable to any vehicle comprising a passenger compartment, in particular, a train, an airplane, a boat, etc.

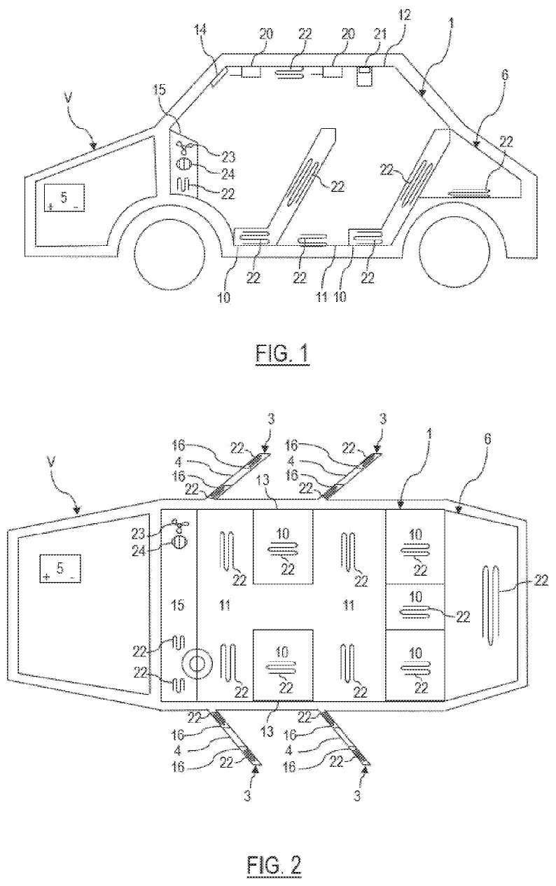

In a known manner and as illustrated in FIGS. 1 and 2, an automobile vehicle V comprises a passenger compartment 1 defined as the part of the vehicle V reserved for users, that is, the living space. The automobile vehicle V comprises a plurality of doors 3 allowing users to get in and out of the vehicle V, that is, to access the passenger compartment 1, a plurality of windows 4 for circulating an air flow between the interior and the exterior of the passenger compartment 1 and a trunk 6 defined as a storage space.

In this example, the passenger compartment 1 comprises a plurality of seats 10, in particular, two front seats and a rear seat. The passenger compartment 1 further comprises various passenger compartment linings to protect and coat the passenger compartment 1, in particular, a floor lining 11, a roof lining 12, and a sidewall lining 13. Preferably, a lining 16 is also present on each door 3. Such linings also provide heat and sound insulation in the passenger compartment 1.

The passenger compartment 1 also comprises a plurality of sun visors 14 allowing users to avoid being dazzled in case of strong external light, an instrument panel 15 located at the front of the automobile vehicle 1 housing various displays and actuators for controlling multimedia, navigation, ventilation and the like. In this example, the automobile vehicle V further comprises a storage space known as a “glove box”.

In a known manner and as illustrated in FIGS. 1 and 2, the automobile vehicle V comprises an electric battery 5 for generating electric current and thus powering the various pieces of electric equipment of the vehicle V, namely the interior and exterior bulbs, radio or electric sockets as an example.

According to the invention, the automobile vehicle V comprises a cleaning system 2 configured to withdraw impurities from the automobile vehicle V.

By virtue of the cleaning system 2, cleaning the vehicle V is carried out internally, which does not require the vehicle to be moved to a cleaning center or an external device to be installed.

Advantageously, as illustrated in FIGS. 1 and 2, the cleaning system 2 is integrated into the passenger compartment 1 in order to be as close as possible to the impurities to be cleaned. Furthermore, this facilitates installation of the cleaning system 2 as well as its maintenance.

Nevertheless, it goes without saying that a part of the cleaning system 2 could not be comprised directly in the passenger compartment 1 but positioned at another place of the automobile vehicle V, in particular, in a rear trunk or in proximity to the engine/motor of the automobile vehicle V.

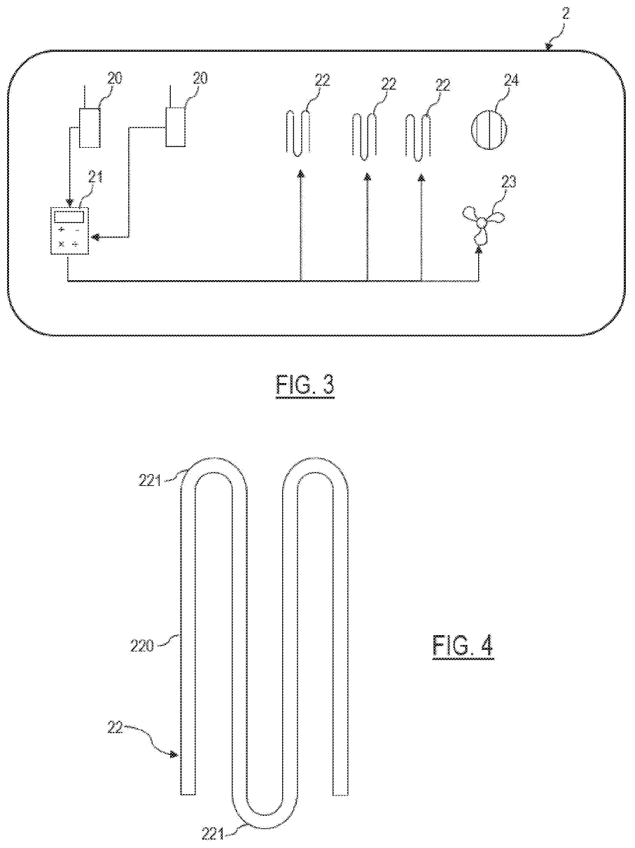

In this example, with reference to FIG. 3, the cleaning system 2 comprises a plurality of measuring members 20, a plurality of heating members 22, a filtering member 24, a ventilation member 23 and a control member 21, the structure and role of which will be described successively hereafter. In this example, the various members 20-24 are housed in the passenger compartment 1 and the various members 20-23 are electrically powered by the electric battery 5. It goes without saying that the members 20-23 could be powered in a different manner.

As illustrated in FIG. 3, the measuring members 20 allow a level of insalubrity N of the passenger compartment 1 to be measured. Advantageously, these measuring members 20 are in the form of gas sensors, such as metal oxide sensors as an example, recording the presence of volatile toxic substances, which are nitrogen dioxide N₁, benzene-toluene-xylene N₂, ozone N₃ and formaldehyde N₄, which makes it possible to determine a level of insalubrity N of the passenger compartment 1. It goes without saying that other types of sensors could be suitable, in particular, to record the presence of volatile toxic substances not mentioned, such as VOCs. Advantageously, the passenger compartment 1 comprises a plurality of measuring members 20, which increases reliability and accuracy of the measurement of the level of insalubrity N. Nevertheless, it goes without saying that the passenger compartment 1 could contain only a single measuring member 20. Moreover, advantageously, the measuring members 20 are located at the front of the passenger compartment 1 in proximity to the driver's position in order to measure insalubrity felt by the driver. Preferably, with reference to FIG. 1, the measuring members 20 are distributed throughout the passenger compartment 1 in order to measure an average representative of the overall level of insalubrity and not a local level of insalubrity of a zone of the passenger compartment 1.

According to the invention, the heating members 22 are able, in use, to produce heat, which is transmitted to the passenger compartment elements 10-16 by conduction and to ambient air by convection. The temperature in the passenger compartment 1 then increases, which has the effect of desorbing the impurities present, that is, extracting the impurities from the passenger compartment elements and turning them into a vapor phase.

The heating members 22 are configured to heat ambient air of the passenger compartment 1 to a temperature above 40° C., preferably above 45° C., more preferably to 50° C., in order to desorb impurities from the passenger compartment 1. Preferably, the heating temperature is below 60° C. so as not to degrade elements of the passenger compartment 1 as will be set forth later.

In this example, with reference to FIG. 4, each heating member 22 is in the form of a planar coil resistor. In other words, each heating member 22 comprises alternating rectilinear cylindrical tubes 220 and angled cylindrical tubes 221. Such a heating member 22 allows a given surface to be heated homogeneously. It goes without saying that the heating members 22 could have a different shape.

According to one aspect of the invention, the heating members 22 are powered in a wired or wireless manner, in particular, by induction. A wireless power supply advantageously allows the passenger compartment element to be made removable from the passenger compartment, for example, a floor mat. Both of these heating methods allow impurities to be desorbed effectively. However, it goes without saying that another heating mode can be chosen.

According to another aspect of the invention, the heating members 22 may be rigid or flexible, the choice being based on their integration into the passenger compartment 1.

According to one aspect of the invention, several passenger compartment elements 10-16 are equipped with a heating member 22 to form a heating passenger compartment element in order to cover the largest possible volume of the passenger compartment 1 subject to impurities. It goes without saying, however, that only one or part of the passenger compartment elements 10-16 can be equipped with a heating member 22, and additionally, that a single passenger compartment element 10-16 can be equipped with a plurality of heating members 22. Passenger compartment elements 10-16 not explicitly mentioned, for the sake of brevity and clarity, can also be equipped with one or more heating members 22. Furthermore, each heating member 22 can be installed in two different ways depending on the installation convenience and use of the passenger compartment element 10-16, namely by insertion/integration into said element or covering the surface of said element. The association of a heating member 22 with a passenger compartment element 10-16 is advantageous since it makes it possible to heat ambient air of the passenger compartment 1 but also to heat internally the passenger compartment element 10-16 with which the heating member 22 is associated in order to thermally desorb impurities contained in said passenger compartment element 10-16.

For example, as illustrated in FIGS. 1 and 2, at least one heating member 22 is inserted in each seat 10, in each sun visor 14, and in the instrument panel 15. Heating members 22 also cover the floor lining 11, roof lining 12, side walls 13, and linings 16 of door 3. In this example, a large part of the passenger compartment elements 10-16 are thus equipped with heating members 22 and form heating passenger compartment elements. For passenger compartment elements 10-16 configured for comfort, for example a seat 10, it is preferable to integrate the heating member 22 in the seat 10 rather than covering it so as not to adversely affect comfort to the users. On the contrary, for the floor lining 11, the heating member 2 can be placed at the surface to reduce costs.

Furthermore, in the example of the preceding paragraph, the trunk 6 is covered with a heating member 22, although it is external to the passenger compartment 1, because it is nevertheless likely to be dirtied by users in the same way as the passenger compartment elements 10-16. For example, the trunk 6 can be dirtied by the users' luggage, by liquid leaks or by a pet. Furthermore, the heating members 22 are integrated into passenger compartment elements 10-16 comprising polyurethane foam or polyvinyl chloride or acrylonitrile butadiene styrene or polypropylene or polyethylene or polyester or an alloy of these thermoplastics, so as to improve heating and desorption. Of course, however, the passenger compartment elements 10-16 can comprise other materials.

As previously indicated, the heating member 22 is configured to heat ambient air in the passenger compartment 1 to a temperature above 40° C. In other words, the exterior temperature of the passenger compartment element 10-16 is above 40° C. This is in contrast to a conventional heated seat the exterior temperature of which is in the range of 30° C. and below 40° C.

The ventilation member 23 allows ambient air and desorbed impurities to be discharged outside the passenger compartment 1. In this example, a single ventilation member 23 is shown in this example, but it goes without saying that a plurality of ventilation members 23 could be used. Preferentially, the ventilation member 23 is located at the instrument panel in order to take advantage of existing ventilation ducts. Preferably, the ventilation member 23 is in the form of a mechanical fan.

The filtering device 24 makes it possible to treat, preferably mechanically or chemically, desorbed impurities before they are discharged outside the passenger compartment 1, for environmental reasons. Such a filtering member 24 is optional. Preferentially, the filtering device 24 is associated with the ventilation member 23 in order to treat and discharge the desorbed impurities simultaneously, but it goes without saying that the filtering device 24 and the ventilation member 23 can be distinct. Preferably, the filtering member 24 is in the form of a chemical filter that destroys molecules of the impurities, for example an ozonolysis or photocatalysis filter, or in the form of a mechanical filter, for example an activated carbon filter. Preferably, the filtering member 24 is placed at the inlet of the ventilation member 23.

The control member 21 is configured to activate the heating members 22 when the level of insalubrity N of the passenger compartment 1 is greater than a predetermined maximum level of insalubrity N_{ET}, that is when the vehicle is in a dirty state POK. Advantageously, the maximum level of insalubrity N_{ET} is determined from data banks on the maximum allowed presence of volatile toxic substances in the passenger compartment 1 of the vehicle V, in order to adopt a comparison scale that is as relevant as possible and adapted to the case of the vehicle V under consideration. However, the maximum level of insalubrity N_{ET} can be determined in a different way. In this example, the control member 21 is in the form of an electronic calculator electrically connected to the measuring members 20, heating members 22 and ventilation member 23 in order to control the cleaning steps.

An example of implementation of a method for cleaning the vehicle V according to the invention will now be set forth.

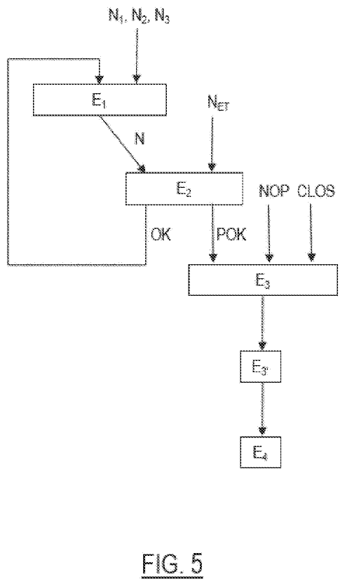

With reference to FIG. 5, the method for cleaning the passenger compartment 1 of the vehicle V comprises a step of measuring E₁ the level of insalubrity N of the passenger compartment 1 by means of the measuring members 20, a step of activating E₂, by the control member 21, the heating members 22 in order to carry out a heating step E₃ at a temperature above 40° C. when the measured level of insalubrity N is greater than a predetermined maximum level of insalubrity N_{ET}.

Preferably, during the measurement step E₁, the measuring members 20 record, at different time instants, the levels of nitrogen dioxide N₁, benzene-toluene-xylene N₂, ozone N₃ and formaldehyde N₄ in the passenger compartment 1 in order to define the level of insalubrity N of said passenger compartment 1. It goes without saying that the measuring members 20 could measure other types of impurities such as VOCs. Preferably, the measurement step E₁ is implemented periodically or each time the automobile vehicle V is inactivated.

In the activation step E₂, the control member 21 compares the measured level of insalubrity N of the passenger compartment 1 at different time instants with the predetermined maximum level of insalubrity N_{ET} and makes it possible to determine accurately when to perform the heating step E₃. Thus, the heating step is activated when the passenger compartment 1 is considered to be in the dirty state POK, that is, when the measured level of insalubrity N is greater than the predetermined maximum level of insalubrity N_{ET}. When the measured level of insalubrity N is lower than the predetermined maximum level of insalubrity N_{ET}, the passenger compartment 1 is considered to be in the clean state OK and no heating is carried out.

According to one aspect of the invention, the heating step E₃ is activated only when the passenger compartment 1 is closed (CLOSED state). For this purpose, the heating system 2 comprises one or more closure sensors (not represented), positioned at the doors 3 or windows 4, in order to determine whether the passenger compartment 1 is closed. Preferably, the closure sensors are electrically connected to the control member 21.

According to another aspect of the invention, the heating step E₃ is activated only in the absence of users (NOP state). For this purpose, the heating system 2 comprises one or more presence sensors (not represented), positioned in the passenger compartment 1, in order to determine whether users are present. Preferably, the presence sensors are electrically connected to the control member 21. The heating step E₃ thus allows for effective treatment, that is, without heat loss and without inconvenience to the vehicle users.

The heating step E₃ is implemented by activating the heating members 22 so that ambient air is at a temperature above 40° C., preferably above 45° C., and even more preferably at 50° C., in order to desorb all the impurities present and thus to allow an effective and not partial cleaning. Preferably, the temperature of the heating members 22 does not exceed 60° C. in order not to impair the passenger compartment elements 10-16. During this step, the heating passenger compartment elements are thus purified of their impurities by the heat released by their heating member 22 transmitted by conduction. As for ambient air, it is also purified by convection of heat from the heating passenger compartment elements. This method therefore makes it possible to advantageously purify the heating passenger compartment elements and ambient air from their impurities, that is the passenger compartment 1 as a whole, which makes the method very effective.

Preferably, the heating step E₃ is implemented for a duration adapted to the level of insalubrity N and the volume of the passenger compartment 1 so as to desorb at least 70% of the impurities and to minimize power consumption. Preferably, the duration is such that all the impurities are desorbed.

With reference to FIG. 5, the method comprises a step of filtering E₃′ the impurities consumed by the filtering member 24 and a step of ventilating E₄ the passenger compartment 1 by the ventilation member 23 in order to discharge ambient air and desorbed impurities outside the passenger compartment 1 by means of the ventilation member 23.

The filtering step E₃′ is implemented by treating the desorbed impurities in order to withdraw possible harmful components therefrom. This step is not essential to the cleaning method of the passenger compartment 1 of the vehicle V but is advantageously carried out for environmental reasons to avoid propagation of potentially harmful desorbed impurities in the atmosphere. This step is carried out once the heating step E₃ has been completed or simultaneously when the ventilation is carried out in a closed circuit.

The ventilation step E₄ is implemented by turning on the ventilation member 23 until the level of insalubrity N of the passenger compartment 1 is lower than the predetermined maximum level of insalubrity N_{ET}. Preferably, the ventilation member 23 operates at a power adapted to the level of insalubrity N and the volume of the passenger compartment so as to rapidly discharge ambient air-suspended impurities outside the passenger compartment 1 via the ventilation member 23, while minimizing power consumption. According to one aspect of the invention, this ventilation step E₄ is carried out once the heating step E₃ has been completed in order to avoid heat losses and during the filtering step E₃′, the desorbed impurities being thus treated at the time of being discharged to outside. However, it goes without saying that this ventilation step E₄ can be started in parallel with the heating step E₂ and after the filtering step E₃′. Advantageously, this step is quick and easy to perform and uses means internal to the vehicle V.

Preferably, in the absence of the filtering step E₃′, the windows 4 of the automobile vehicle V are open during the ventilation step E₄.

Advantageously, this method makes it possible to determine accurately when to perform cleaning of the passenger compartment 1, thus avoiding a waste of time and money related to too early a cleaning and the discontent of the users of the vehicle V related to too late a cleaning. This method also has the advantage of not requiring vehicle movement or manpower to be performed, being internal to the vehicle V, which makes it less expensive. Such a cleaning method is perfectly adapted to a vehicle with a high occupancy rate.

1-10. (canceled)

11. A vehicle comprising a passenger compartment, wherein a cleaning system for the passenger compartment comprising at least one member for measuring the level of insalubrity of said passenger compartment, at least one heating member configured to heat ambient air of the passenger compartment to a temperature above 40° C. in order to desorb impurities from the passenger compartment, at least one ventilation member configured to discharge ambient air and desorbed impurities outside the passenger compartment and at least one control member configured to activate the heating member if the measured level of insalubrity is greater than a predetermined maximum level of insalubrity, said vehicle comprising at least one passenger compartment element comprising at least one heating member configured to heat the passenger compartment element to a temperature above 40° C. in order to form a heating passenger compartment element to desorb impurities from the passenger compartment element.

12. The vehicle according to claim 11, wherein the heating member is configured to heat ambient air of the passenger compartment to a temperature above 45° C., 50° C.

13. The vehicle according to claim 11, wherein the heating member is configured to heat ambient air of the passenger compartment to a temperature below 60° C.

14. The vehicle according to claim 11, wherein the heating member is internally integrated into the passenger compartment element.

15. The vehicle according to claim 11, wherein the heating member is positioned at the surface of the passenger compartment element.

16. The vehicle, according to claim 11, wherein at least one heating passenger compartment element is a seat.

17. The vehicle according to claim 11, wherein at least one heating passenger compartment element is a passenger compartment lining.

18. The vehicle according to claim 11, wherein at least one heating passenger compartment element is a floor lining.

19. The vehicle according to claim 11, wherein at least one heating passenger compartment element is the instrument panel.

20. The vehicle according to claim 11, wherein the cleaning system comprises at least one filtering member configured to treat the desorbed impurities when discharged to outside of the passenger compartment.

21. A method for cleaning the passenger compartment of the vehicle according to claim 11, comprising:
a step of measuring the level of insalubrity of the passenger compartment by means of the measuring member,
a step of activating the heating step when the measured level of insalubrity is greater than a predetermined maximum level of insalubrity by means of the control member,
a step of heating the passenger compartment in order to heat ambient air of the passenger compartment and thus to desorb impurities present by means of the heating member and
a step of ventilating the passenger compartment in order to discharge ambient air and desorbed impurities outside the passenger compartment by means of the ventilation member.
